(19) United States
(12) Patent Application Publication
(10) Pub. No.: US 2017/0348363 A1
(43) Pub. Date: Dec. 7, 2017

(54) IMMUNE MODULATION FOR ISCHEMIA DISEASE AND ACCELERATION OF RECOVERY AFTER ISCHEMIC EVENTS BY MICROBIOME STIMULATED T REGULATORY CELLS

(71) Applicant: Biotech Holdings, LLC, San Diego, CA (US)

(72) Inventors: Thomas Ichim, San Diego, CA (US); Santosh Kesari, Santa Monica, CA (US); Jessica Baker, Encinitas, CA (US)

(21) Appl. No.: 15/611,699

(22) Filed: Jun. 1, 2017

Related U.S. Application Data
(60) Provisional application No. 62/343,901, filed on Jun. 1, 2016.

(51) Int. Cl. A61K 35/747; A61K 35/744; A61K 35/15; A61K 35/745; A61K 45/06; C12N 5/078

(52) U.S. Cl. CPC A61K 35/747; A61K 45/06; C12N 5/0634; A61K 35/15; A61K 35/745; A61K 35/744; C12N 2501/00

(57) ABSTRACT

Disclosed are compositions of matter treatment protocols and combination therapies aimed at stimulating neoangiogensis in ischemia tissue and means of acceleration host ischemic healing. In one embodiment antibiotics are administered to a patient in need of therapy, said antibiotics of endogenious microflora. Subsequently probiotic and or probiotic and prebiotic are administered to generate “natural” microflora. Said reconstituted microflora provides a means for generation of cardiac self-reactive T regulatory cells capable of stimulating angiogenesis and or myocardial protection.

### CROSS-REFERENCE TO RELATED APPLICATIONS

This application claims priority to U.S. Provisional Application No. 62/343,901, filed Jun. 1, 2016, which is incorporated herein by reference in its entirety.

### FIELD OF THE INVENTION

The invention pertains to the field of therapeutic angiogenesis and regeneration of ischemic tissue, furthermore the invention pertains to the use of ex vivo expanding of T regulatory cells generated after treatment of a patient with a probiotic mixture

### SUMMARY

The teachings herein relate to methods of treating ischemic tissue damage comprising the steps of: a) identifying a patient with ischemic tissue; b) optimizing microflora in said patient; c) assessing immune cells in said patient capable of modulating ischemic damage; d) modulating said immune cells to be amplified by said microflora so as to reduce ischemic damage.

### DETAILED DESCRIPTION OF THE INVENTION

The invention teaches the use of probiotic mixtures to induce in vivo expansion of T regulatory cells. Said T regulatory cells are capable in vivo of stimulation angiogenesis in one aspect of the invention. In another aspect, T regulatory cells generated in vivo after probiotic administration are expanded ex vivo by culture conditions including it-2, cd3, tgf, it-10 and other known means in the art. In one embodiment, subsequent to in vivo expansion, said T regulatory cells are isolated and expand more proficiently in vivo.

In one embodiment, specific probiotic bacteria are administered individually or in combination with other bacteria. The lactic acid bacterium and/or Bifidobacterium that are used in accordance with the present invention may comprise from 10(6) to 10(12) colony forming units (CFU) of bacteria per gram of support material, and more particularly from 10(8) to 10(12) CFU of bacteria/gram of support material, preferably 10(9) to 10(12) CFU/gram of support material for the lyophilized form.

Suitably the lactic acid bacterium and/or Bifidobacterium used in accordance with the present invention may be administered at a dosage of from about 10.sup.6 to about 10.sup.12 CFU of microorganism/dose, preferably about 10.sup.8 to about 10.sup.12 CFU of microorganism/dose. By the term “per dose” it is meant that this amount of microorganism is provided to a subject either per day or per intake, preferably per day. For example, if the microorganism is to be administered in a food product (for example in a yogurt)—then the yogurt will preferably contain from about 10.sup.8 to 10.sup.12 CFU of the microorganism. Alternatively, however, this amount of microorganism may be split into multiple administrations each consisting of a smaller amount of microbial loading—so long as the overall amount of microorganism received by the subject in any specific time (for instance each 24 hour period) is from about 10.sup.6 to about 10.sup.12 CFU of microorganism, preferably 10.sup.8 to about 10.sup.12 CFU of microorganism.

In accordance with the present invention an effective amount of at least one strain of a microorganism may be at least 10.sup.6 CFU of microorganism/dose, preferably from about 10.sup.6 to about 10.sup.12 CFU of microorganism/dose, preferably about 10.sup.8 to about 10.sup.12 CFU of microorganism/dose. In one embodiment, preferably the lactic acid bacterium and/or Bifidobacterium used in accordance with the present invention (such as a strain of Lactobacillus spp.; for example a strain of Lactobacillus acidophilus, Lactobacillus salivarius and/or Lactobacillus plantarum and/or a strain of Bifidobacterium spp., such as a strain of Lactobacillus acidophilus or Lactobacillus salivarius, for example Lactobacillus acidophilus strain such as NCFM or Lactobacillus salivarius strain 33) such as a strain of Bifidobacterium animalis subsp. lactis, for example Bifidobacterium animalis subsp. lactis strain 420 (B420)) may be administered at a dosage of from about 10.sup.6 to about 10.sup.12 CFU of microorganism/day, preferably about 10.sup.8 to about 10.sup.12 CFU of microorganism/day. Hence, the effective amount in this embodiment may be from about 10.sup.6 to about 10.sup.12 CFU of microorganism/day, preferably about 10.sup.8 to about 10.sup.12 CFU of microorganism/day.

Disclosed are methods, diagnostic means, and compositions of matter and therapeutics for the diagnosis and treatment of pregnancy complications through immune modulation of a mammal in need. In one embodiment the invention provides assays for establishing risk of pregnancy complications, said assays being utilized to provide therapeutic interventions to ensure successful pregnancies. Pregnancy complications include RSA, preterm birth, pre-eclampsia including HELP, premature rupture of the membrane, Antepartum hemorrhage including placental abruption, chorioamnionitis, Intrauterine growth restriction, placenta pravaevia, sequalae of intraamniotic infection.

The probiotic mixtures may be used according to the present invention in the form of solid or liquid preparations or alternatives thereof. Examples of solid preparations include, but are not limited to tablets, capsules, dusts, granules and powders which may be wettable, spray-dried or freeze-dried. Examples of liquid preparations include, but are not limited to, aqueous, organic or aqueous-organic solutions, suspensions and emulsions. Suitable examples of forms include one or more of: tablets, pills, capsules, ovules, solutions or suspensions, which may contain flavoring or coloring agents, for immediate-, delayed-, modified-, sustained-, pulsed- or controlled-release applications. By way of example, if the composition of the present invention is used in a tablet form—such for use as a functional ingredient—the tablets may also contain one or more of: excipients such as microcrystalline cellulose, lactose, sodium citrate, calcium carbonate, dibasic calcium phosphate and glycine; disintegrants such as starch (preferably corn, potato or tapioca starch), sodium starch glycoliate, croscarmellose sodium and certain complex silicates; granulation binders such as polyvinylpyrrolidone, hydroxypropylmethylcellulose (HPMC), hydroxypropylcellulose (HPC), sucrose, gelatin and acacia; lubricating agents such as magnesium stearate, stearic acid, glyceryl behenate and talc may be included. Furthermore, examples of nutritionally acceptable carriers for use in preparing the forms include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid monoglycerides and diglycerides, petroethral fatty acid esters, hydroxymethylcellulose, polyvinylpyrrolidone, and the like. Preferred excipients for the forms include lactose, starch, a cellulose, milk sugar or high molecular weight polyethylene glycols.

In one embodiment, probiotic bacteria are administered in the form of a nutraceutical. Nutraceuticals, whether in the form of a liquid extract or dry composition, are edible and may be eaten directly by humans or mammals. Said nutraceuticals are preferably provided to humans in the form of additives or nutritional supplements e.g., in the form of tablets of the kind sold in health food stores, or as ingredients in edible solids, more preferably processed food products such as cereals, breads, tofu, cookies, ice cream, cakes, potato chips, pretzels, cheese, etc., and in drinkable liquids e.g., beverages such as milk, soda, sports drinks, and fruit juices. Thus, in one embodiment a method is provided for enhancing the nutritional value of a food or beverage by intermixing the food or beverage with a nutraceutical in an amount that is effective to enhance the nutritional and probiotic or immune modulatory value of the food or beverage. In one embodiment, a flavoring agent is added. Preferred flavoring agents include sweeteners such as sugar, corn syrup, fructose, dextrose, maltodextrose, cyclamates, saccharin, phenyl-alanine, xylitol, sorbitol, maltitol, and herbal sweeteners such as Stevia. Examples of foods into which probiotics useful for the practice of the invention can be incorporated into include soft drinks, a fruit juice or a beverage comprising whey protein, health teas, cocoa drinks, milk drinks and lactic acid bacteria drinks.

The nutraceuticals described herein are intended for human consumption and thus the processes for obtaining them are preferably conducted in accordance with Good Manufacturing Practices (GMP) and any applicable government regulations governing such processes. Especially preferred processes utilize only naturally derived solvents. In contrast to nutraceuticals, the so-called “medical foods” are not meant to be used by the general public and are not available in stores or supermarkets. Medical foods are not those foods included within a healthy diet to decrease the risk of disease, such as reduced-fat foods or low-sodium foods, nor are they weight loss products. A physician prescribes a medical food when a patient has special nutrient needs in order to manage a disease or health condition, and the patient is under the physician's ongoing care. The label must clearly state that the product is intended to be used to manage a specific medical disorder or condition. An example of a medical food is nutritionally diverse medical food designed to provide targeted nutritional support for patients with chronic inflammatory conditions.

Subsenquent to probiotic administration Treg are expanded ex vivo from PMBC and subsequently injected. T regulatory cells are an essential component of the immune system protecting the body against autoimmune attack. This is illustrated by early studies in which neonatally thymectomized mice suffered from systemic autoimmunity, which were rescued by transfer of CD4 cells. Subsequent studies identified the T regulatory (Treg) phenotype as possessing the IL-2 receptor CD25, which is somewhat problematic given that this receptor is found on activated T cells as well. Peripheral blood contains a small population of T cell lymphocytes that express the T regulatory phenotype (“Treg”), i.e., positive for both CD4 and CD25 antigens. There are several subsets of Treg cells. One subset of regulatory cells develops in the thymus. Thymic derived Treg cells function by a cytokine-independent mechanism, which involves cell to cell contact. They are essential for the induction and maintenance of self-tolerance and for the prevention of autoimmunity (Shevach, 2000 Annu. Rev. Immunol. 18: 423-449). These regulatory cells prevent the activation and proliferation of autoreactive T cells that have escaped thymic deletion or recognize extrathymic antigens, thus they are critical for homeostasis and immune regulation, as well as for protecting the host against the development of autoimmunity. Thus, immune regulatory CD4.sup.+CD25.sup.+ T cells are often referred to as “professional suppressor cells.”

Naturally arising CD4.sup.+CD25.sup.+Treg cells are a distinct cell population of cells that are positively selected on high affinity ligands in the thymus and that have been shown to play an important role in the establishment and maintenance of immunological tolerance to self antigens. Deficiencies in the development and/or function of these cells have been associated with severe autoimmunity in humans and various animal models of congenital or induced autoimmunity.

Treg cells manifest their tolerogenic effects directly via cell-to-cell contact or indirectly via soluble factors. Although the suppressive mechanisms of these cells remain to be fully elucidated, blockade of IL-2 expression in effector T cells (Teff), physical elimination of Teff cells, induction of tolerogenic dendritic cells (DCs) via CTLA-4/B7 axis, and inhibition of Teff cells via TGF-.beta. and IL-10 are some of the mechanisms that have been implicated to date. It also has been shown that reverse signaling through CTLA-4/CD80 into Teff cells plays an important role in their inhibition by Treg cells. Similarly, interactions between CTLA-4 on Treg cells and CD80 on DCs can result in reverse signaling and upregulation of the indoleamine dioxygenase enzyme that is involved in tolerance via the regulation of tryptophan metabolism.

Treg cells can also be generated by the activation of mature, peripheral CD4.sup.+ T cells. Studies have indicated that peripherally derived Treg cells mediate their inhibitory activities by producing immunosuppressive cytokines, such as transforming growth factor-beta (TGF-.beta.) and IL-10 (Kingsley et al., 2002 J. Immunol. 168: 1080; Nakamura et al., 2001 J. Exp. Med. 194: 629-644). Treg are have been described in the literature as being hypoproliferative in vitro (Sakaguchi, 2004 Ann. Rev. Immunol. 22: 531). Trenado et al. provided the first evaluation of the therapeutic efficacy of ex vivo activated and expanded CD4.sup.+CD25.sup.+regulatory cells in an in vivo mouse model of disease (Trenado et al., 2002 J. Clin. Invest. 112(11): 1688-1696). To date, all known activities of T regulatory cells have been related to immune modulation

The invention provides means of utilizing T regulatory cells for stimulation of angiogenesis in ischemic tissue. In one embodiment the invention teaches the use of non-matched cord blood derived T regulatory cells for treatment of ischemic conditions. The present invention includes compositions and methods for expanding T regulatory cells (Tregs), natural T regulatory cells (nTregs) for use in stimulation of angiogenesis. More preferably, the expanded cells retain nTreg or Treg phenotype and angiogenic activity following expansion.

The invention provides compositions and methods for expanding Tregs, preferably nTregs, without the subsequent reversion of the nTregs to T effector cells. Accordingly, such an expansion methodology allows for the establishment of a cell bank useful for stimulation of angiogenesis. In one aspect of the present invention, nTreg expansion can be performed by isolating nTregs from a desired cell source and subsequently culture expanding the cells in the presence of a primary signal and a co-stimulatory signal. Agents useful for stimulating a primary signal and an a co-stimulatory signal on Tregs may be used in soluble form, attached to the surface of a cell, or immobilized on a surface as described herein. In a preferred embodiment both primary and co-stimulatory agents are co-immobilized on a surface, for example a bead or an engineered cell. In one embodiment, the molecule providing the primary activation signal, such as a CD3 ligand, and the co-stimulatory molecule, such as a CD28 ligand are coupled to or loaded on the same surface, for example, a particle or an engineered cell. Said cells can be administered alone or with angiogenic cells to treat ischemic disorders

In another embodiment, the invention provides a method of expanding Tregs, preferably nTregs to unprecedented numbers using a repetitive stimulation procedure. In one embodiment, the method of expanding nTregs comprises restimulating nTregs based upon cell size. Preferably, nTregs exhibiting a cell size about the size of a resting nTreg are chosen for restimulation. In some instances, the size of a resting nTreg is about 8.5.mu.m. That is, the invention is based on the discovery that cell size is a parameter that contributes to the success of expanding nTregs without losing nTreg phenotype and suppressor activity. In another embodiment, the method of expanding nTregs comprises restimulating nTregs in the presence of Rapamycin. Preferably, nTregs isolated from peripheral blood is re-stimulated in the presence of Rapamycin. That is, the invention is based on the discovery that Rapamycin contributes to the success of expanding nTregs isolated from peripheral blood without losing nTreg phenotype and suppressor activity. Preferably, the expanded cells of the invention maintain Foxp3 profile indicative of nTregs. In one embodiment, the population of expanded nTregs expresses specific natural Treg markers such as Foxp3 and Latency Associated Peptide (LAP), displayed Treg specific demethylation in the Foxp3 gene, and contain very few IL-2, IFN.gamma., IL-17 secreting cells. The expanded cells of the invention also are able to suppress limb loss in a xenogenic model of critical limb ischemia. In other embodiments, at least a portion of the active cell population is stored for later implantation/infusion. The population may be divided into more than one aliquot or unit such that part of the population of nTregs is retained for later application while part is applied immediately to the patient. Moderate to long-term storage of all or part of the cells in a cell bank is also within the scope of this invention. For the purpose of the invention, Treg and nTreg may be interchangeable.

### Example 1

Wild type 8-10 week old virgin CBA/J female mice orally administered a probiotic mixture of L. acidophilus, L. casei, Lactobacillus reuteri, Bifidobacterium bifidium, and Streptococcus thermophiles at a dosage of 5×10⁸ cfu/day. Another 10 mice were used as controls and treated with saline. CD4 CD25 expressing cells from donor mice, either probiotic treated (Treated) or Control (Saline) were administered in ischemic limb following the mouse model of CLI published by Murphy et al. J Transl Medicine.

| Mouse | | | | | |
|---|---|---|---|---|---|
| Number | Day 7 | Day 14 | Day 21 | Day 28 | Day 35 |
| 1 Control | | | | Limb Loss | |
| 2 Control | | | | | Limb Loss |
| 3 Control | | | | Limb Loss | |
| 4 Control | | | Limb Loss | | |
| 5 Control | | | | Limb Loss | |
| 6 Control | | | | Limb Loss | |
| 7 Control | | | | Limb Loss | |
| 8 Treated | | | | | |
| 9 Treated | | | | | |
| 10 Treated | | | | | |
| 11 Treated | | | | | |
| 12 Treated | | | | | |
| 13 Treated | | | | | |
| 14 Treated | | | | | |

1. A method of treating ischemic tissue damage comprising the steps of: a) identifying a patient with ischemic tissue; b) optimizing microflora in said patient; c) assessing immune cells in said patient capable of modulating ischemic damage; d) modulating said immune cells to be amplified by said microflora so as to reduce ischemic damage.

2. The method of claim 1, wherein said ischemia tissue associated pathology is comprised of pathologies selected from a group comprising of: a) global cerebral ischemia; b) stroke; c) seizure; d) stimulant withdraw syndrome; e) opiate withdraw; f) ischemic heart disease; g) cognitive dysfunction; h) spinal cord injury; i) lack of motor function; j) paralysis; k) female sexual dysfunction associate with ischemia; l) erectile dysfunction; m) spontaneous recurrent abortion; n) osteonecrosis; o) osteoarthrosis; p) neoplasia; q) COPD;

3. The method of claim 1, wherein said optimization of microflora is performed by methods comprising of; a) probiotic; b) prebiotic; c) fecal transplant; d) apple cider vinegar; e) yogurt administration; f) tea tree oil; f) peppermint extract; g) excessive water intake; h)

4. The method of claim 1, wherein said immune cells selected from a group comprised of; a) cells expressing Foxp3; b) CD4+CD25+ cells; c) cells expressing TGF-Beta

5. The method of claim 4, wherein said cells are autologous.

6. The method of claim 4, wherein said cells are allogeneic.

7. The method of claim 1, wherein said patient is treated with an antibiotic, subsequent to which a probiotic mixture is administered and at the time of probiotic engraftment leukocytes are extracted and cells expressing foxp3.

8. The method of claim 7, wherein said foxp3 expressing cells are injecting directly in an ischemic tissue.

9. The method of claim 8, wherein said cells are treated with an anti-inflammatory agent.

10. The method of claim 9, wherein said ant-inflammatory agent is selected from a group comprising of: BLC, Eotaxin-1, Eotaxin-2, G-CSF, GM-CSF, 1-309, ICAM-1, IFN-gamma, IL-1 alpha, IL-1 beta, IL-1 ra, IL-2, IL-4, IL-5, IL-6, IL-6 sR, IL-7, IL-8, IL-10, IL-11, IL-12 p40, IL-12 p70, IL-13, IL-15, IL-16, IL-17, MCP-1, M-CSF, MIG, MIP-1 alpha, MIP-1 beta, MIP-1 delta, PDGF-BB, RANTES, TIMP-1, TIMP-2, TNF alpha, TNF beta, sTNFRI, sTNFRIIAR, BDNF, bFGF, BMP-4, BMP-5, BMP-7, b-NGF, EGF, EGFR, EG-VEGF, FGF-4, FGF-7, GDF-15, GDNF, Growth Hormone, HB-EGF, HGF, IGFBP-1, IGFBP-2, IGFBP-3, IGFBP-4, IGFBP-6, IGF-1, Insulin, M-CSF R, NGF R, NT-3, NT-4, Osteoprotegerin, PDGF-AA, PIGF, SCF, SCF R, TGFalpha, TGF beta 1, TGF beta 3, VEGF, VEGFR2, VEGFR3, VEGF-D 6Ckine, Axl, BTC, CCL28, CTACK, CXCL16, ENA-78, Eotaxin-3, GCP-2, GRO, HCC-1, HCC-4, IL-9, IL-17F, IL-18 BPa, IL-28A, IL-29, IL-31, IP-10, I-TAC, LIF, Light, Lymphotactin, MCP-2, MCP-3, MCP-4, MDC, MIF, MIP-3 alpha, MIP-3 beta, MPIF-1, MSPalpha, NAP-2, Osteopontin, PARC, PF4, SDF-1 alpha, TARC, TECK, TSLP 4-1BB, ALCAM, B7-1, BCMA, CD14, CD30, CD40 Ligand, CEACAM-1, DR6, Dtk, Endoglin, ErbB3, E-Selectin, Fas, Flt-3L, GITR, HVEM, ICAM-3, IL-1 R4, IL-1 R1, IL-10 Rbeta, IL-17R, IL-2Rgamma, IL-21R, LIMPII, Lipocalin-2, L-Selectin, LYVE-1, MICA, MICB, NRG1-beta1, PDGF Rbeta, PECAM-1, RAGE, TIM-1, TRAIL R3, Trappin-2, uPAR, VCAM-1, XEDARActivin A, AgRP, Angiogenin, Angiopoietin 1, Angiostatin, Catheprin S, CD40, Cripto-1, DAN, DKK-1, E-Cadherin, EpCAM, Fas Ligand, Fcg RIIB/C, Follistatin, Galectin-7, ICAM-2, IL-13 R1, IL-13R2, IL-17B, IL-2 Ra, IL-2 Rb, IL-23, LAP, NrCAM, PAI-1, PDGF-AB, Resistin, SDF-1 beta, sgp130, ShhN, Siglec-5, ST2, TGF beta 2, Tie-2, TPO, TRAIL R4, TREM-1, VEGF-C, VEGFR1Adiponectin, Adipsin, AFP, ANGPTL4, B2M, BCAM, CA125, CA15-3, CEA, CRP, ErbB2, Follistatin, FSH, GRO alpha, beta HCG, IGF-1 sR, IL-1 sRII, IL-3, IL-18 Rb, IL-21, Leptin, MMP-1, MMP-2, MMP-3, MMP-8, MMP-9, MMP-10, MMP-13, NCAM-1, Nidogen-1, NSE, OSM, Procalcitonin, Prolactin, PSA, Siglec-9, TACE, Thyroglobulin, TIMP-4, TSH2B4, ADAM-9, Angiopoietin 2, APRIL, BMP-2, BMP-9, C5a, Cathepsin L, CD200, CD97, Chemerin, DcR3, FABP2, FAP, FGF-19, Galectin-3, HGF R, IFN-gammalpha/beta ?R2, IGF-2, IGF-2 R, IL-1R6, IL-24, IL-33, Kallikrein 14, Legumain, LOX-1, MBL, Neprilysin, Notch-1, NOV, Osteoactivin, PD-1, PGRP-5, Serpin A4, sFRP-3, Thrombomodulin, TLR2, TRAIL R1, Transferrin, WIF-1ACE-2, Albumin, AMICA, Angiopoietin 4, BAFF, CA19-9, CD163, Clusterin, CRTAM, CXCL14, Cystatin C, Decorin, Dkk-3, DLL1, Fetuin A, aFGF, FOLR1, Furin, GASP-1, GASP-2, GCSF R, HAI-2, IL-17B R, IL-27, LAG-3, LDL R, Pepsinogen I, RBP4, SOST, Syndecan-1, TACI, TFPI, TSP-1, TRAIL R2, TRANCE, Troponin I, uPA, VE-Cadherin, WISP-1, and RANK.
